(19) United States
(12) Patent Application Publication
(10) Pub. No.: US 2020/0033371 A1
(43) Pub. Date: Jan. 30, 2020

(54) MEASURING METHOD AND MEASURING DEVICE

(71) Applicant: Educational foundation Kyushu Bunka Gakuen, Nagasaki (JP)

(72) Inventor: Kazuhiro Ichikawa, Nagasaki (JP)

(21) Appl. No.: 16/513,104

(22) Filed: Jul. 16, 2019

(30) Foreign Application Priority Data
Jul. 17, 2018 (JP) 2018-134309
Jul. 12, 2019 (JP) 2019-130425

(51) Int. Cl. G01N 33/94; G01N 33/543; G01R 33/36

(52) U.S. Cl. CPC G01N 33/9406; G01R 33/3635; G01N 33/54326

(57) ABSTRACT

Provided is a method of measuring a condition of neurotransmission in a target by means of a magnetic resonance process in which electron spin is used. The method includes a first step of applying a magnetic resonance process to the target containing therein a contrast medium containing molecules responsive to electric potential, to thereby obtain magnetic resonance signals, and a second step of determining a condition of neurotransmission in the target in accordance with the magnetic resonance signals having been obtained in the first step.

### FIELD OF THE INVENTION

The present invention relates to a method of measuring a condition of neurotransmission, and further to an apparatus for doing the same.

### BACKGROUND ART

A nerve cell, on receipt of an electric signal, varies a membrane potential for controlling sodium channel and opening/closing of sodium channel to thereby transmit the electric signal to an end of an axon. Transmission of the electric signal to an end of an axon causes neurotransmitter to be released, and the thus released neurotransmitter excites an adjacent nerve cell to thereby allow the adjacent nerve cell to receive the electric signal. The electric signal is transmitted in such a manner to thereby cause neurotransmission.

As an example of the variation in a membrane potential, there is known the study in which a neural axon of doryteuthis is used (for instance, see the non-patent document 1). FIG. 8 illustrates an example showing how a membrane potential varies, and FIG. 9 illustrates propagation of electric potential caused by neural activity, namely, variation in time and space in a gigantic axon of doryteuthis. It is understood that fluctuation in a membrane potential caused by opening/closing of channel is about 100 mV in a time frame of a few milliseconds.

As a method of measuring a membrane potential, there is known a method of measuring a membrane potential by combining a minute electrode to a cell (for instance, see the patent document 1).

### PRIOR ART DOCUMENTS

### Patent Document

[PATENT DOCUMENT 1] Japanese Patent Application Publication No. 1109 (1997)-024031

### Non-Patent Document

[NON-PATENT DOCUMENT 1] Physical chemistry for those studying chemistry and life science, Reymond Chang, Tokyo Kagaku Doujin

[NON-PATENT DOCUMENT 2] Analytical chemistry, (US), 2014, vol. 86, No. 2, p. 1045-1052

[NON-PATENT DOCUMENT 3] Jornal of Mgnetic Resonance, (NL), 2010, vol. 202, No.2, p. 267-273

### DISCLOSURE OF THE INVENTION

### Problems to be Solved by the Invention

The measured neurotransmission condition can be applied to visualization of pain transmission and pharmacometrics to analgestic activity.

However, a conventional method of measuring an electric potential is accompanied with limitations in application, such as invasiveness caused by insertion of an electrode, and capability of measurement only in a position where a probe is arranged. Furthermore, a conventional method of measuring an electric potential by means of a probe is accompanied with a problem that it is difficult to measure an electric potential at a deep point in organism. The object of the present invention is to provide a method of measuring a condition of neurotransmission, which is not invasive, and is capable of carrying out the measurement even at a deep point in organism, and further, to provide an apparatus for doing the same.

### Solution to the Problems

The inventor has studied and researched in order to solve the problems as mentioned above, and has found that the invention identified below met with the above-mentioned object, and thus, the present invention was made.

That is, the present invention provides the followings.

<1> A method of measuring a condition of neurotransmission in a target by means of a magnetic resonance process in which electron spin is used, the method including a first step of applying a magnetic resonance process to the target containing therein a contrast medium containing molecules responsive to electric potential, to thereby obtain magnetic resonance signals, and a second step of determining a condition of neurotransmission in the target in accordance with the magnetic resonance signals having been obtained in the first step.

<2> The method as set forth in <1>, wherein the second step includes a step of imaging the magnetic resonance signals having been obtained in the first step.

<3> The method as set forth in <1> or <2>, wherein the magnetic resonance process is comprised of one of an electron spin resonance process and an Overhauser MRI.

<4> The method as set forth in any one of <1> to <3>, wherein the molecules responsive to electric potential are defined by the general formula (1) identified below.

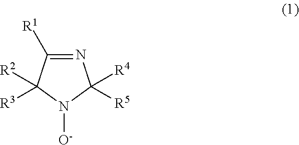

In the general formula (1), R¹ indicates one of groups selected from groups consisting of an alkyl group, a phenyl group, an amino group, and an azacycloalkyl group, R² and R³ each independently indicates an alkyl group, and R⁴ and R⁵ each independently indicates one of groups selected from groups consisting of an alkyl group, a phenyl group, and an alkyl carboxyl group.

<5> An apparatus for measuring a condition of neurotransmission in a target by means of a magnetic resonance process in which electron spin is used, the apparatus including signal obtaining means for applying a magnetic resonance process to the target containing therein a contrast medium containing molecules responsive to electric potential, to thereby obtain magnetic resonance signals, and neurotransmission condition judging means for determining a condition of neurotransmission in the target in accordance with the magnetic resonance signals having been obtained by the signal obtaining means.

### Advantages Obtained by the Invention

In accordance with the present invention, there is provided a method of measuring a condition of neurotransmission, which is not invasive, and is capable of carrying out the measurement even at a deep point in organism, and further provided an apparatus for doing the same.

### BRIEF DESCRIPTION OF THE DRAWINGS

FIG. 1 illustrates the concept of a method of measuring a condition of neurotransmission, in accordance with the present invention.

FIG. 2 is a schematic view showing integral EPR spectral translation and how an image intensity changes, caused by fluctuation of an electric potential.

FIG. 3 illustrates differential type EPR spectrums caused by fluctuation of an electrical potential.

FIG. 4 illustrates an OMRI image at the equilibrium potential.

FIG. 5 illustrates how an electron spin resonance spectrum intensity changes with the lapse of time due to fluctuation in an electric potential in a solution of molecules responsive to an electric potential.

FIG. 6 illustrates the apparatus 1 for carrying out measurement, in accordance with the present invention.

FIG. 7 illustrates the apparatus 2 for carrying out measurement, in accordance with the present invention.

FIG. 8 illustrates an example of fluctuation in a membrane potential.

FIG. 9 illustrates fluctuation in time and space in an axon.

### EMBODIMENTS FOR REDUCING THE INVENTION TO PRACTICE

Preferred embodiments in accordance with the present invention will be explained in detail hereinbelow. Elements of the invention identified below are just examples (typical example) of the embodiments in accordance with the present invention. It should be noted that the scope of the present invention is not to be limited to the explanation made below, unless the gist of the present invention is changed.

The method of measuring a condition of neurotransmission in a target by means of a magnetic resonance process in which electron spin is used, in accordance with the present invention, includes a first step of applying a magnetic resonance process to the target containing therein a contrast medium containing molecules responsive to electric potential, to thereby obtain magnetic resonance signals, and a second step of determining a condition of neurotransmission in the target in accordance with the magnetic resonance signals having been obtained in the first step.

The method in accordance with the present invention has the following features.

(1) A magnetic resonance process is used.

(2) A contrast medium for detecting fluctuation in an electric potential is used.

(3) Fluctuation in an electric potential is detected by virtue of spectral fluctuation.

(4) Propagation of electric potential fluctuation in a target is measured or imaged.

By using a contrast medium containing therein molecules responsive to an electric potential, the molecules responsive to an electric potential follow fluctuation in a membrane potential caused by signal transmission in neurotransmission, to thereby change condition thereof. By applying a magnetic resonance process to a target containing therein a contrast medium containing therein molecules responsive to an electric potential, various magnetic resonance signals are detected in dependence on the condition of molecules responsive to an electric potential, caused in dependence on fluctuation in a membrane potential, and thus, it is possible to grasp the magnetic resonance signals as a condition of neurotransmission.

Since the method in accordance with the present invention uses both a magnetic resonance process and a contrast medium containing therein molecules responsive to an electric potential, the method is theoretically capable of realizing a neurotransmission mapping of a whole of organism and a deep point in organism. Consequently, the method in accordance with the present invention can be applied to visualization in pain transmission and/or pharmacometrics to analgestic activity as well as to tactile sense and all of occasional exercises. Expected ripple effect is very high.

In the method in accordance with the present invention, it is preferable that the second step includes a step of imaging the magnetic resonance signals having been obtained in the first step. By imaging a condition of neurotransmission, it is possible to readily grasp a condition of spatial neurotransmission in a target.

The method in accordance with the present invention may be used as a method of imaging neurotransmission in real time in terminal stimulation in organism. The present invention provides a completely new method of realizing visualization in real time in neurotransmission in terminal stimulation in target organism. Expected target includes visualization in pain transmission, and pharmacometrics to analgestic activity. Furthermore, the method and the apparatus both in accordance with the present invention makes it possible to theoretically wholly measure neurotransmission in a deep point in organism as a whole, ensuring that ripple effect is broadly expected.

In the method in accordance with the present invention, it is preferable that the magnetic resonance process is comprised of an electron spin resonance process or an Overhauser MRI. By using these magnetic resonance processes, it is possible to obtain more accurate images.

It is more preferable that the magnetic resonance process is comprised of an electron spin resonance process (ESR or EPR). It is preferable that a magnetic field has an intensity equal to or smaller than 20 mT.

It is preferable that the magnetic resonance process is comprised of a pulse process. It is possible in a pulse process to carry out measurement in a period of time in the range of about hundreds of microseconds to about 1 millisecond both inclusive, ensuring it possible to measure dynamic fluctuation in activity propagation.

In the method in accordance with the present invention, it is preferable that the molecules responsive to electric potential are defined by the general formula (1) identified below. In the molecules defined with the general formula (1), fluctuation following electric potential fluctuation tends to be caused at a millisecond level, and thus, the molecules may be preferably used as molecules responsive to electric potential in the method in accordance with the present invention.

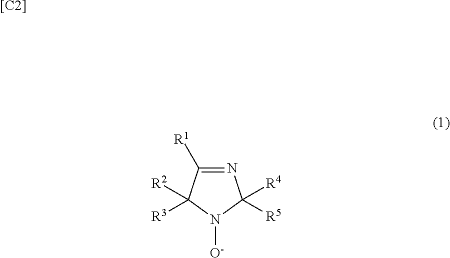

In the general formula (1), R¹ indicates one of groups selected from groups consisting of an alkyl group, a phenyl group, an amino group, and an azacycloalkyl group, R² and R³ each independently indicates an alkyl group, and R⁴ and R⁵ each independently indicates one of groups selected from groups consisting of an alkyl group, a phenyl group, and an alkyl carboxyl group.

The present invention further provides an apparatus for measuring a condition of neurotransmission in a target by means of a magnetic resonance process in which electron spin is used, the apparatus including signal obtaining means for applying a magnetic resonance process to the target containing therein a contrast medium containing molecules responsive to electric potential, to thereby obtain magnetic resonance signals, and neurotransmission condition judging means for determining a condition of neurotransmission in the target in accordance with the magnetic resonance signals having been obtained by the signal obtaining means.

Furthermore, the apparatus in accordance with the present invention is able to realizing visualization of neurotransmission in terminal stimulation in organism in real time.

The apparatus in accordance with the present invention is suitable for carrying out the method in accordance with the present invention.

[Method in Accordance with the Present Invention]

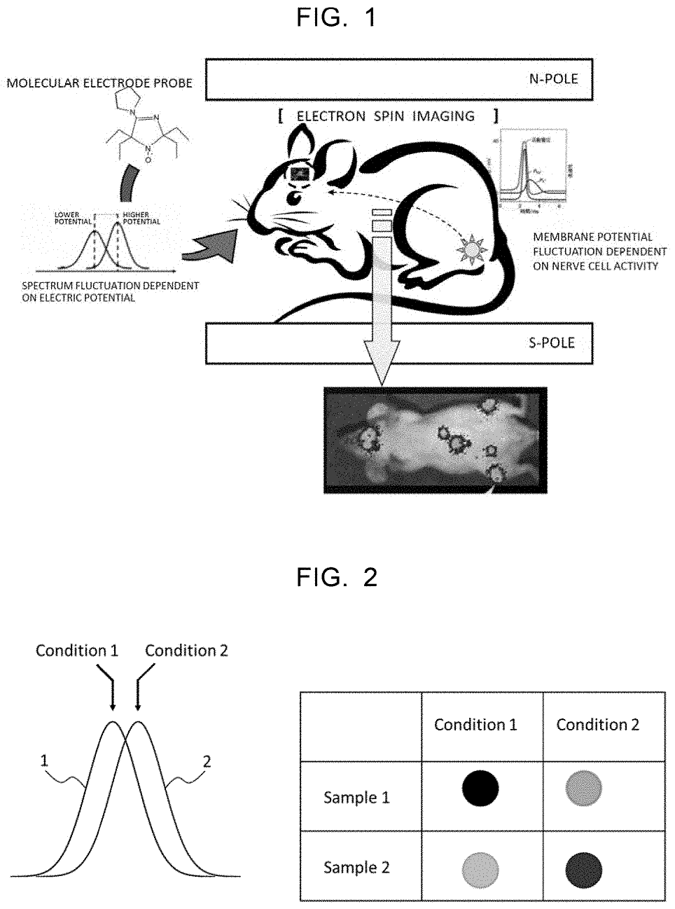

FIG. 1 illustrates the concept of a method of measuring a condition of neurotransmission, in accordance with the present invention. In the method in accordance with the present invention, there is used a magnetic resonance process (for instance, pulse ESR technique or and OMI apparatus) capable of measuring and imaging at a time scale in the range of about a microsecond to about a millisecond by virtue of a membrane potential response of a molecule responsive to an electric potential or “a molecular electrode” probe in organism, thereby accomplishing measuring and imaging of neural activity and neurotransmission process. Furthermore, it is possible to carry out pharmacometrics in a disease model, and development of molecular electrode derivatives by utilizing the method in accordance with the present invention. It is quite difficult to accomplish imaging an electric potential at milliseconds from the point of view of sensitivity. Imaging an electric potential at milliseconds can be accomplished only by the high-speed technique of the inventor. Thus, the present invention cannot be readily reached or accomplished by the others. The method in accordance with the present invention makes it possible to carry out measurement at a high speed to thereby measure a dynamic electric potential at a high speed, thus contributing to imaging neural activity.

In the method in accordance with the present invention, both molecules responsive to an electric potential and a magnetic resonance process are used to thereby obtain fluctuation in magnetic resonance signals caused in dependence on fluctuation in a membrane potential in a target, to thereby measure a condition of neurotransmission.

A membrane potential indicates a difference in an electric potential between inside and outside of a cell. As mentioned above, a nerve cell varies a membrane potential for controlling a sodium channel or opening/closing of a sodium channel to thereby transmit signals to a terminal in an axon. As mentioned above, electric potential fluctuation at above 120 mV is generated at a time frame of about 5 milliseconds in potential transmission caused by neural activity. In order to measure and visualize this phenomenon at a deep point in organism, it is necessary to prepare molecules responsive to an electric potential, having a response speed equal to or smaller than 1 second (preferably, at an order of millisecond), and to have the technique for measurement and visualization.

### (Target to be Measured)

A target to be measured by the method in accordance with the present invention is an example or organism (not to be limited to a mouse and so on). Specifically, a target includes organism such as human being, cow, horse, pig, monkey, marmot, rabbit, rat, and mouse. A target may be limited to cow, horse, pig, monkey, marmot, rabbit, rat, and mouse, other than human being. As an alternative, a target may include cell and organ other than organism. After a contrast medium containing therein molecules responsive to an electronic potential was administered or dropped into a target, the method in accordance with the present invention is carried out. For instance, in the case that a target is comprised of organism, a method of administering a contrast medium to a target (an administration route) is not to be limited to any specific method. A route for administering a contrast medium is comprised preferably of an intravenous injection, application to a skin, administration into an abdominal cavity or subcutaneous administration.

### (Contrast Medium)

A contrast medium to be used in the method in accordance with the present invention includes molecules responsive to an electric potential. Molecules responsive to an electric potential mean molecules having a reversibly variable structure in dependence on fluctuation in an electric potential.

Molecules responsive to an electric potential, to be used in the present invention, are used as “a molecular electrode” probe. The molecules have an unpaired electron, and a molecular structure thereof varies in dependence on fluctuation in a membrane potential in a target, causing a condition of the unpaired electron to be varied.

Specifically, it is preferable to select molecules responsive to an electric potential, having a potential switching speed equal to smaller than 1 second. Herein, a potential switching speed indicates a speed at which a structure of a molecule varies due to fluctuation in an electric potential. A potential switching speed can be measured by a conventional process such as a magnetic resonance process.

In order to measure a condition of neurotransmission further accurately, it is preferable that a potential switching speed is equal to or smaller than millisecond level, and it is more preferable that a potential switching speed is equal to or smaller than 500 milliseconds. A potential switching speed may be set equal to or smaller than 300 milliseconds, 200 milliseconds, 100 milliseconds, 80 milliseconds, 50 milliseconds, 30 milliseconds, 10 milliseconds, 5 milliseconds or 1 millisecond. As an alternative, a potential switching speed may be set to a microsecond level, and thus, may be set equal to or greater than 50 microseconds, 100 microseconds or 500 microseconds.

FIG. 2 is a schematic view showing integral EPR spectral translation and how an image intensity changes, caused by fluctuation of an electric potential. For instance, it is supposed that an example A (for instance, a molecule having the structure A shown in the formula (2) identified below) has one (1) electric potential prior to bonding with a positive ion, and that an example B (for instance, a molecule having the structure B shown in the formula (2) identified below) has two (2) electric potential after having bonded with a positive ion. EPR spectrum of an example having 1 electric potential (spectrum 1 of sample 1 in FIG. 2) is turned by virtue of electric potential fluctuation into EPR spectrum of an example having 2 electric potential (spectrum 2 of sample 2 in FIG. 2). If measurement and imaging were carried out at a maximum spectrum absorption point (Condition 1 or Condition 2 in FIG. 2), intensity fluctuation shown in the table in FIG. 2 is detected, and thus, an electric potential can be imaged. By using a contrast medium containing therein molecules responsive to an electric potential, it is possible to measure fluctuation in position of electron spin resonance (ESR) spectrum which is dependent on an electric potential.

In the method in accordance with the present invention, a structure of a molecule responsive to an electric potential is not to be limited to any specific structure. A molecule defined with the general formula (1) identified below is preferably used as a molecule responsive to an electric potential to be used in the method in accordance with the present invention. A molecule defined with the general formula (1) varies a structure thereof by virtue of fluctuation in an electric potential, and shows various magnetic resonance actions. Furthermore, since the molecule readily varies a structure thereof at a millisecond level by virtue of fluctuation in an electric potential, the molecule is preferably used for detecting fluctuation in a membrane potential.

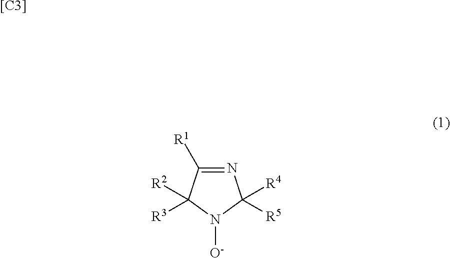

In the general formula (1), R¹ indicates one of groups selected from groups consisting of an alkyl group, a phenyl group, an amino group, and an azacycloalkyl group, R² and R³ each independently indicates an alkyl group, and R⁴ and R⁵ each independently indicates one of groups selected from groups consisting of an alkyl group, a phenyl group, and an alkyl carboxyl group.

An alkyl group may be of a straight chain, bifurcated or cyclic. As an alkyl group, there may be used an alkyl group having a carbon number in the range of 1 to 8 or 1 to 5 both inclusive. Specifically, there may be used a methyl group or an ethyl group.

A phenyl group may be non-substituted or have a substituent.

An amino group is a functional group expressed with “—NR⁶R⁷”, wherein R⁶ and R⁷ each independently indicates an alkyl group having a carbon number in the range of 1 to 8 or 1 to 5 both inclusive. Specifically, an amino group, a dimethyl amino group, a diethyl amino group, a methyl ethyl amino group may be used.

An azacycloalkyl group is a function group in which a carbon atom in a cycloalkyl group is replaced with a nitrogen atom. An azacycloalkyl group may have membered rings in the range of 4 to 10 or 5 to 8 both inclusive. It is preferable that a nitrogen atom in an azacycloalkyl group is bonded to a carbon atom having an imidazoline skeleton. Specifically, a pyrrolidyl group, a piperidyl group, an azepanyl group or an azocanyl group may be used.

An alkyl carboxyl group is a functional group in which an alkyl group expressed with “—R⁸—COOH” and a carboxyl group is bonded to each other. An alkyl group having a carbon number in the range of 1 to 8 or 1 to 5 both inclusive may be selected as R⁸.

In the general formula (1), it is preferable that R¹ indicates one of groups selected from groups consisting of an alkyl group, a phenyl group, and an azacycloalkyl group, and it is more preferable that R¹ indicates an azacycloalkyl group.

In the general formula (1), it is preferable that R⁴ and R⁵ each independently indicates an alkyl group or a phenyl group, and it is more preferable that R⁴ and R⁵ each indicates an alkyl group.

In a molecule defined with the general formula (1), as shown with the formula (2) identified below, a positive ion (X⁺) can be chemically or coordinately bonded with a third nitrogen atom present in an imidazoline ring. By desorption of a positive ion to a third nitrogen atom present in an imidazoline ring, an electron density in a first nitrogen atom to which an oxygen radical is to be bonded varies (see the dotted line in the formula (2)), resulting in that three positions at which electron spin resonance absorption occurs, derived from nitrogen nucleus varies, vary. It is possible to measure an electric potential by virtue of this phenomenon.

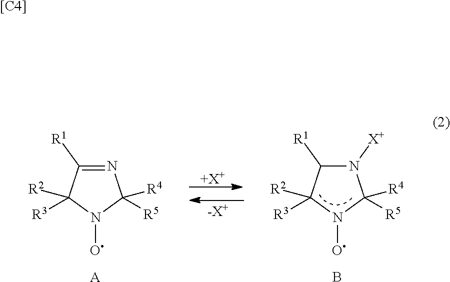

As a molecule defined with the general formula (1), there may be used (4-amino-2, 2, 5, 5-tetraethyl-2-imidazoline-1-yloxyl) radical, (4-(pyrrolidine-1-yl)-2, 2, 5, 5-tetraethyl-2-imidazoline-1-yloxyl) radical, (4-(azepan-1-yl)-2, 2, 5, 5-tetraethyl-2-imidazoline-1-yloxyl) radical or the molecules listed in the non-patent documents 2 and 3.

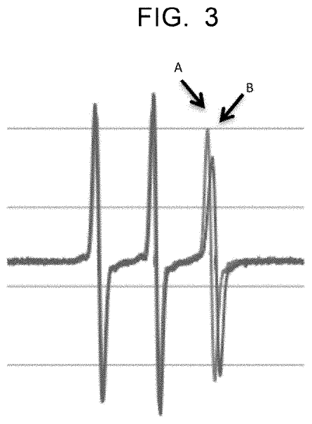

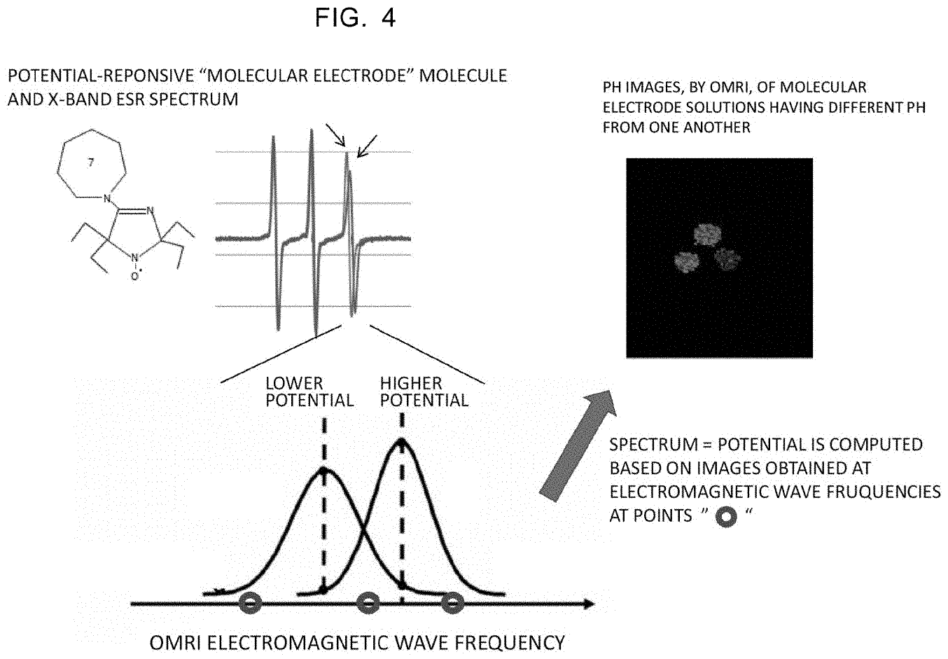

FIGS. 3 and 4 show the result of the experiment relating to the present invention.

In FIG. 3, differential type EPR spectrums obtained by measuring two examples in a static condition thereof, each having an electric potential different from each other, are overlapped each other. FIG. 3 illustrates differential type EPR spectrums caused by fluctuation of an electrical potential. The measurement was carried out in the conditions identified below.

### Preparation of the Example:

As a molecule responsive to an electric potential, expressed with the general formula (1), there was used 4-amino-2, 2, 5, 5-tetramethyl-3-imidazoline-1-yloxyl radical. Dissolving 4-amino-2, 2, 5, 5-tetramethyl-3-imidazoline-1-yloxyl radical in phosphoric acid buffer solution such that the final concentration was 100 μM, and then, the resultant solution was set to pH 6 or 8.0 by means of hydrochloric acid solution and sodium hydroxide solution. Thus, as examples each having an electric potential different from each other, there were obtained 4-amino-2, 2, 5, 5-tetramethyl-3-imidazoline-1-yloxyl radical (corresponding to the structure A identified in the above-mentioned general formula (2)), and 4-amino-2, 2, 5, 5-tetramethyl-imidazoline-1-yloxyl radical (corresponding to the structure B identified in the above-mentioned general formula (2)).

### Conditions for Measuring X-Band:

JEOL X-band ESR JES-REIX, an intensity of a magnetic field 3330 Gauss, sweeping width +/−5 mT, sweeping time 10 seconds, magnetic field modulation 100 kHz, 0.05 mT, microwave 9.45 GHz, 1 mW Magnetic field location of spectrum:

Since spectrum was obtained in a magnetic field sweeping mode, a frequency was constant.

As illustrated in FIG. 3, as an electric potential fluctuates, X-band EPR spectrum varies to the spectrum B from the spectrum A. A magnetic field location at the right of the spectrum A is 3345 GHz, and a magnetic field location at the right of the spectrum B is 3345.7 GHz.

FIG. 4 illustrates the results of the experiment with respect to spectrum imaging of a solution containing molecules responsive to an electric potential. By carrying out spectrum imaging of a solution containing molecules responsive to an electric potential, having an electric potential different from each other, an electric potential was imaged with respect to (static) phenomenon having occurred in about a few seconds. In OMRI image at an equilibrium potential, illustrated in FIG. 4, the differences among the solutions are 0, 90 and 250 mV from the top in a counter-clockwise direction.

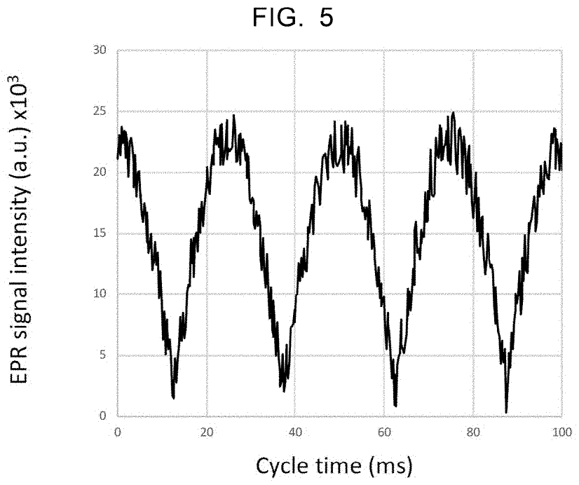

FIG. 5 illustrates how an intensity of electron spin resonance spectrum varies with the lapse of time, due to fluctuation in an electric potential in a solution containing molecules responsive to an electric potential. The measurement was carried out with the following conditions.

### Preparation of the Example:

As a molecule responsive to an electric potential, expressed with the general formula (1), there was used 4-amino-2, 2, 5, 5-tetramethyl-2-imidazoline-1-yloxyl radical. Dissolving 4-amino-2, 2, 5, 5-tetramethyl-2-imidazoline-1-yloxyl radical in phosphoric acid buffer solution such that the final concentration was 100 μM, and then, the resultant solution was set to pH 6 by means of hydrochloric acid solution and sodium hydroxide solution.

### Conditions for Measuring X-Band:

JEOL X-band ESR JES-REIX, an intensity of a magnetic field 3345 Gauss, fixed, magnetic field modulation 100 kHz, 0.05 mT, microwave 9.45 GHz, 1 mW

### Steps in the Experiment:

A sine-curve voltage having a peak/peak voltage of 1 V was input at a frequency of about 60 Hz from an external oscillator (NF WF1973) to an electrode electrically connected to an X-band solution cell, to thereby modulate a solution potential. ESR output data was obtained at a sampling rate of about 50 kHz in line with the modulation.

Δ pH1 corresponds to about 50 mV. By applying 1 V at a maximum spectral absorption location, which is an initial location, in a pH 6 solution, the potential was modulated at a point at which about pH 9, where the potential response reached at plateau, and hence, was no longer varied, was sufficiently over, to thereby measure data fluctuation.

As illustrated in FIG. 5, as a solution potential varies, it is observed that a signal intensity accordingly decreases (transfer to non-resonance absorption from maximum resonance absorption) or increases (transfer to maximum resonance absorption from non-resonance absorption). This indicates that a molecule responsive to an electric potential responds to potential fluctuation at a millisecond level to thereby provide data about an electric potential of a solution. It was not reported that a molecule responsive to an electric potential responds to high-speed fluctuation of an electric potential, and that such response could be visualized. The inventor has first discovered that the molecule had high responsiveness, and thus, followed potential fluctuation at a millisecond level with the result of the spectral fluctuation.

It is possible to obtain potential data of a contrast medium having responded to potential fluctuation at a millisecond level, by measuring ESR at a millisecond level through the use of a molecule responsive to an electric potential, and further, successively measuring EPR.

[Apparatus in Accordance with the Present Invention]

The apparatus in accordance with the present invention is designed to include an apparatus for measuring magnetic resonance, a control processing section, and a display section. The apparatus for measuring magnetic resonance acts as signal obtaining means, and the control processing section acts as neurotransmission condition judging means. Magnetic resonance signals having been obtained by the apparatus for measuring magnetic resonance are analyzed in the control processing section to thereby obtain neurotransmission data.

The apparatus for measuring magnetic resonance may be comprised of an ESR apparatus or an Overhauser MRI apparatus. The ESR apparatus may be of a CW type or a pulse type. It is preferable that the ESR apparatus is of a pulse type because a period of time necessary for measurement can be shortened. As the OMI apparatus, there may be used the apparatus disclosed in WO2010/110384. That is, there may be used “an apparatus including magnetic field generating means for generating a magnetic field by which magnetic resonance is to be excited in a target, moving means for moving the target in a magnetic field generated by the magnetic field generating means, by moving the target or the magnetic field generating means, measurement means for applying a gradient magnetic field, without stopping while moving by the moving means, in a direction “y” in which the target moves relative to the magnetic field generating means and/or a direction “x” perpendicular to the direction “y” to thereby image signals of the target by means of a phase encoder and/or a frequency encoder, and correction means for correcting the image signals to remove influence exerted by the movement in the direction “y”, to thereby have corrected image signals”.

The control processing section applies Fourier transformation and imaging process to data derived from magnetic resonance signals obtained by the apparatus for measuring magnetic resonance, to thereby detect a condition of neurotransmission. The thus obtained data can be memorized into the control processing section, and data necessary for analysis can be read out of the control processing section.

Furthermore, the control processing section stores therein a control program for controlling measurement conditions to be applied to the apparatus for measuring magnetic resonance. The control processing section can output a control program, and control measurement conditions to be applied to the apparatus for measuring magnetic resonance.

The display section displays ESR spectrum, NMR spectrum both obtained by the control processing section, and analyzed images.

### [Method 1 for Measurement]

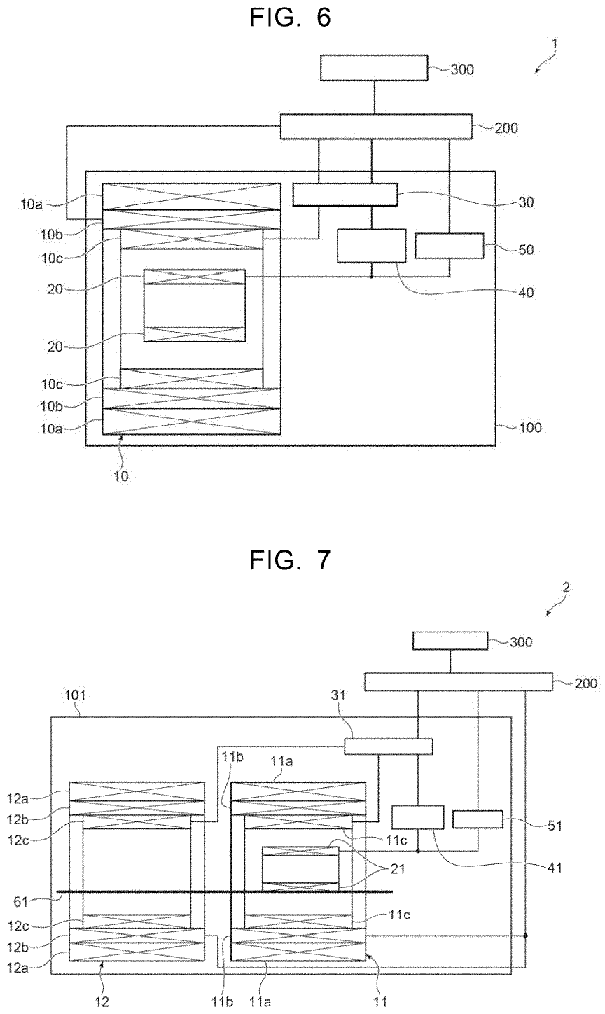

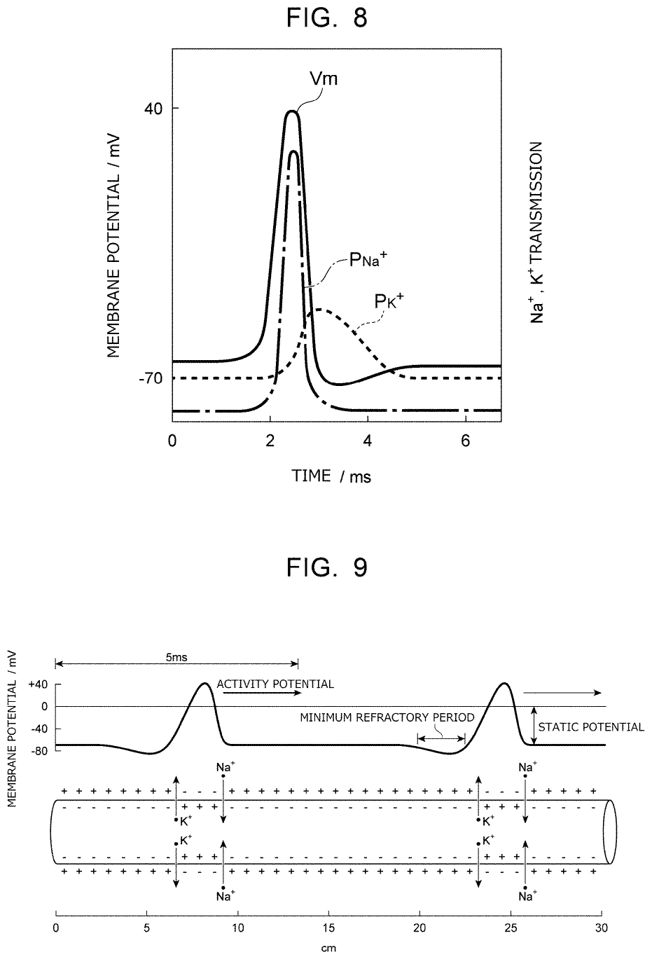

The method 1 in accordance with the present invention is an example to be carried out through the use of the measurement apparatus 1 (see FIG. 6) including a pulse type ESR apparatus 100, a control processing section 200, and a display section 300.

The pulse type ESR apparatus 100 includes an external magnetic field generator 10 comprising an eternal magnet 10a, a magnetic field sweeping coil 10b, and a magnetic field gradient coil 10c, an RF coil (a resonator) 20, a pulse generator 30, an RF pulse generator 40, and a detector 50.

The magnetic field sweeping coil 10b and the detector 50 are electrically connected to the control processing section, and are controlled by instructions transmitted from the control processing section 200. The pulse generator 30 is electrically connected to the control processing section 200, the magnetic field gradient coil 10c and the RF pulse generator 40. The magnetic field gradient coil 10c and the RF pulse generator 40 are controlled by a pulse sequence generated by the pulse generator 30 in accordance with instructions received from the control processing section 200. The display section 300 is electrically connected to the control processing section 200, and displays analysis data having been processed in the control processing section 200.

### First Step

There is generated a predetermined static magnetic field having an intensity greater than 0, but equal to or smaller than 50 mT (for instance, 20 mT) by controlling the magnetic field sweeping coil 10b. There is determined a pulse sequence for measurement, based on sites to be measured, S/N ratio, and so on. A period of time for measurement is set, for instance, equal to or smaller than 1 second (100 microseconds to 1 millisecond, 1 millisecond to 10 milliseconds or 10 to 100 milliseconds, for instance).

First, a mouse as a target to be measured is put in the RF coil 20. A contrast medium containing therein molecules A responsive to an electric potential has been already administered to the target mouse. It should be noted that a part of a mouse such as a head and a tail can be determined as a target. Then, RF pulses (a frequency is in the range of about 500 to about 600 MHz both inclusive) generated in accordance with a predetermined pulse sequence is irradiated to the mouse lying in the RF coil 20. The magnetic field gradient coil 10c is driven in accordance with the predetermined pulse sequence to thereby generate predetermined times a gradient magnetic field having a predetermined intensity. ESR signals caused by electron spin resonance are detected by the detector 50.

The measurement is successively carried out at a predetermined interval with the result that ESR signals can be obtained over time. An interval at which the measurement is carried out is set equal to or smaller than 1 second (for instance, 10 to 100 milliseconds or 10 to 500 milliseconds), for instance.

### Second Step

The control processing section 200 applies Fourier transformation to ESR signals having been detected in the first step to thereby extract and analyze data relating to a range of a magnetic field including a location at which maximum spectrum resonance absorption of the molecule A responsive to an electric potential is found. A location at which maximum spectrum resonance absorption of the molecule A responsive to an electric potential is found in a magnetic field can be identified with data having been obtained by measuring ESR spectrum of the molecule A and stored in the control processing section 200.

The molecule A responsive to an electric potential varies a structure thereof by virtue of potential fluctuation of a membrane potential, and thus, an absorption intensity (a signal intensity) at a location at which maximum spectrum resonance absorption of the molecule A responsive to an electric potential is found decreases. By analyzing the absorption intensity of the molecule A over time, it is possible to identify a part of the molecule A in which the absorption intensity is low, ensuring visualization of pain. By analyzing a direction in which a part of the molecule A in which the absorption intensity is low moves, it is possible to judge the direction as a direction in which a nerve runs. In addition, it is possible to judge that neurotransmission is carried out in a part in which reduction of an absorption intensity of the molecule A is observed.

ESR signals may be visualized. A condition of neurotransmission can be visualized by virtue of image fluctuation (fluctuation of a part in which a signal intensity is low) over time.

In the method 1, the first step may be carried out with a part to be measured in a target being changed (with a position of the RF coil 20 being changed to collect data of ESR signals). As a result, useful data about neurotransmission can be obtained.

In the method 1, the first step may be carried out simultaneously at two or more points in a direction in which a nerve runs. By carrying out the first step simultaneously at two or more points, it is possible to obtain useful data about neurotransmission by virtue of data synchrony (the same data is measured after fixed delay).

In the method 1, it is possible to obtain data about a location. When a specific part is to be measured, it is sometimes necessary to finish the measurement in a reduced period of time. In the case that reduction in a period of time for the measurement is given priority to obtaining data about a location in dependence on a part to be measured and a purpose of measurement, it is possible to carry out the measurement with the magnetic field gradient coil being off.

In the case that reduction in a period of time for the measurement is given priority to obtaining data about a location, the method in accordance with the present invention may be carried out by means of a measurement apparatus including a pulse type ESR apparatus designed not to include a magnetic field gradient coil. For instance, the measurement may be carried out by means of a measurement apparatus including an ESR apparatus including an external magnetic field generator having an eternal magnet and a magnetic field sweeping coil, an RF coil (a resonator), a pulse generator, an RF pulse generator, and a detector, a control processing section, and a display section. The method for measurement is identical with the method 1 except that the steps relating to a magnetic field gradient coil are not carried out.

Even in the case that a magnetic field gradient coil is turned off, or an apparatus including no magnetic field gradient coil is used, the measurement is carried out on varying points to be measured in a target (for instance, the measurement is carried out at a plurality of points over a head to a tail of a mouse), ensuring that it is possible to distinguish parts in which fluctuation in an absorption intensity of the molecules A responsive to an electric potential is observed, from parts in which the fluctuation is not observed.

Furthermore, by carrying out the measurement simultaneously at two or more points, it is possible to estimate neurotransmission by virtue of data synchrony (the same data is measured after fixed delay).

### [Method 2 for Measurement]

The method 2 in accordance with the present invention is an example to be carried out through the use of the measurement apparatus 2 (see FIG. 7) including an OMRI (Overhauser MRI) apparatus 101, a control processing section 200, and a display section 300.

The OMRI apparatus 101 includes a first external magnetic field generator 11 comprising an eternal magnet 11a, a magnetic field sweeping coil 11b, and a magnetic field gradient coil 11c, a second external magnetic field generator 12 comprising an eternal magnet 12a, a magnetic field sweeping coil 12b, and a magnetic field gradient coil 12c, an RF coil (a resonator) 21, a pulse generator 31, an RF pulse generator 41, a detector 51, and a carrier 61.

The magnetic field sweeping coil 11b, the magnetic field sweeping coil 12b and the detector 51 are electrically connected to the control processing section, and are controlled by instructions transmitted from the control processing section 200.

The pulse generator 31 is electrically connected to the control processing section 200, the magnetic field gradient coil 11c, the magnetic field gradient coil 12c and the RF pulse generator 41. The magnetic field gradient coil 11c, the magnetic field gradient coil 12c and the RF pulse generator 41 are controlled by a pulse sequence generated by the pulse generator 31 in accordance with instructions received from the control processing section 200. The carrier 61 moves the RF coil 21 or the first and second external magnetic field generators to ensure the RF coil 21 to move in turn in magnetic fields generated by the first and second external magnetic field generators.

The display section 300 is electrically connected to the control processing section 200, and displays analysis data having been processed in the control processing section 200.

### First Step

A magnetic field to be generated by the first external magnetic field generator 11 is set to have an intensity greater than 0, but equal to or smaller than 50 mT (for instance, 20 mT), and further, to be a static magnetic field. A magnetic field to be generated by the second external magnetic field generator 12 is set to have an intensity greater than the intensity of a magnetic field generated by the first external magnetic field generator 11 (for instance, 1.5 T), and the intensity is set to be greater than 0, but equal to or smaller than 11 T. There is determined a pulse sequence for measurement, based on sites to be measured, S/N ratio, and so on. A period of time for measuring nuclear magnetic resonance (NMR) is set, for instance, equal to or smaller than 1 second (100 microseconds to 1 millisecond, 1 to 10 milliseconds or 10 to 100 milliseconds, for instance).

First, the RF coil 21 in which a mouse is put is positioned in a magnetic field generated by the first external magnetic field generator 11. A contrast medium containing therein molecules A responsive to an electric potential has been already administered to the target mouse.

Then, RF pulses (a frequency is in the range of about 500 to about 600 MHz both inclusive) generated in accordance with a predetermined pulse sequence is irradiated to the mouse lying in the RF coil 20 to thereby excite electron spin.

After the electron spin has been excited, the RF coil 21 is moved by the carrier 62 into a magnetic field generated by the second external magnetic field generator 12. The RF coil 21 is moved in a few seconds (for instance, 1 to 10 seconds).

After the RF coil 21 was moved into a magnetic field generated by the second external magnetic field generator 12, the RF coil 21 applies RF pulses (a frequency is in the range of 700 to 800 MHz both inclusive) to the mouse to thereby cause nuclear magnetic resonance. Furthermore, the magnetic field gradient coil 12c is driven in accordance with a predetermined pulse sequence to thereby generate predetermined times a gradient magnetic field having a predetermined intensity. NMR signals caused by nuclear magnetic resonance are detected by the detector 51.

The RF coil 21 is moved by the carrier 62 into the first external magnetic field generator 11, and then, the same steps as mentioned above are repeated to thereby obtain NMR signals over time.

### Second Step

The control processing section 200 applies Fourier transformation to NMR signals having been detected in the first step to thereby extract and analyze data relating to a range of a magnetic field including a location at which maximum spectrum resonance absorption of the molecule A responsive to an electric potential is found. Similarly to the method 1, it is possible to have data about neurotransmission by analyzing the fluctuation in an absorption intensity of the molecules A responsive to an electric potential. NMR signals may be visualized. A condition of neurotransmission can be visualized by virtue of image fluctuation with the lapse of time.

In the method 2, similarly to the method 1, the first step may be carried out with a part to be measured in a target being changed. As an alternative, the measurement in the method may be carried out simultaneously at two or more points.

The method in accordance with the present invention is not to be limited to the methods 1 and 2.

For instance, the first step in the method in accordance with the present invention may be designed to be a step in which, by using the measurement apparatus illustrated in FIG. 9, after electron spin was excited in a magnetic field generated by the first external magnetic field generator, RF pulses (a frequency is in the range of 700 to 800 MHz both inclusive) may be applied to a mouse in a magnetic field generated by the first external magnetic field generator without moving the RF coil to thereby cause nuclear magnetic resonance.

As an alternative, a measurement apparatus including a CW type ESR apparatus in place of a pulse type ESR apparatus may be used to carry out the measurement to a target containing therein a contrast medium containing molecules responsive to an electric potential. In case of measurement through the use of a measurement apparatus including a CW type ESR apparatus, the first step may be a step of measurement in which a magnetic field is swept over a certain range while irradiating predetermined microwaves.

As an alternative, the first step in the method in accordance with the present invention may be designed to be a step in which, by a measurement apparatus including a CW type ESR apparatus, magnetic fields are fixed at 2 to 5 points including a location at which maximum spectrum resonance absorption is found in a magnetic field in molecules responsive to an electric potential, contained in a target, and irradiation of microwaves and detection of ESR signals are carried out at a predetermined interval (for instance, one second or shorter, or 10 to 100 milliseconds).

The measurement apparatus in accordance with the present invention is not to be limited to the measurement apparatuses 1 and 2. The measurement apparatus to be used in the present invention may be selected in dependence on a target and a method to be used. For instance, an apparatus including no magnetic field gradient coil. Furthermore, there may be used an apparatus in which a magnetic field generator is comprised of an electromagnet. For instance, an electromagnet may be used in place of the eternal magnet 10a in ESR apparatus 100, and an electromagnet may be used in place of the eternal magnets 11a and 12a in OMRI apparatus 101.

### INDUSTRIAL APPLICABILITY

The measurement method and apparatus in accordance with the present invention makes it possible to wholly measure neurotransmission at a deep point theoretically wholly in organism. A wide ripple effect may be expected.

### INDICATION BY REFERENCE NUMERALS

1, 2 Measurement apparatus in accordance with the present invention

10 External magnetic field generator

11 First external magnetic field generator

12 Second external magnetic field generator

10a, 11a, 12a Eternal magnet

10b, 11b, 12b Magnetic field sweeping coil

10c, 11c, 12c Magnetic field gradient coil

20, 21 RF coil

30, 31 Pulse generator

40, 41 RF pulse generator

50, 51 Detector

61 Carrier

100 ESR apparatus

101 OMRI apparatus

200 Control processing section

300 Display section

1. A method of measuring a condition of neurotransmission in a target by means of a magnetic resonance process in which electron spin is used, the method including:
a first step of applying a magnetic resonance process to the target containing therein a contrast medium containing molecules responsive to electric potential, to thereby obtain magnetic resonance signals; and
a second step of determining a condition of neurotransmission in the target in accordance with the magnetic resonance signals having been obtained in the first step.

2. The method as set forth in claim 1, wherein the second step includes a step of imaging the magnetic resonance signals having been obtained in the first step.

3. The method as set forth in claim 1, wherein the magnetic resonance process is comprised of one of an electron spin resonance process and an Overhauser

4. The method as set forth in claim 1, wherein the molecules responsive to electric potential are defined by the general formula (1) identified below. 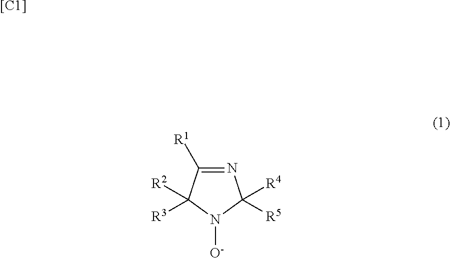
In the general formula (1),
R¹ indicates one of groups selected from groups consisting of an alkyl group, a phenyl group, an amino group, and an azacycloalkyl group,
R² and R³ each independently indicates an alkyl group, and
R⁴ and R⁵ each independently indicates one of groups selected from groups consisting of an alkyl group, a phenyl group, and an alkyl carboxyl group.

5. An apparatus for measuring a condition of neurotransmission in a target by means of a magnetic resonance process in which electron spin is used, the apparatus including:
signal obtaining means for applying a magnetic resonance process to the target containing therein a contrast medium containing molecules responsive to electric potential, to thereby obtain magnetic resonance signals; and
neurotransmission condition judging means for determining a condition of neurotransmission in the target in accordance with the magnetic resonance signals having been obtained by the signal obtaining means.

6. The method as set forth in claim 2, wherein the magnetic resonance process is comprised of one of an electron spin resonance process and an Overhauser MRI.

7. The method as set forth in claim 2, wherein the molecules responsive to electric potential are defined by the general formula (1) identified below. 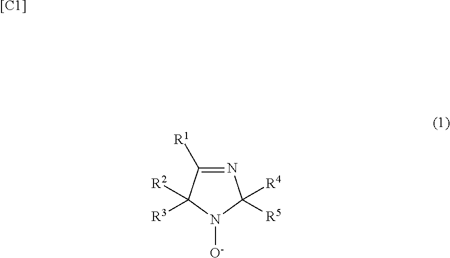
In the general formula (1),
R¹ indicates one of groups selected from groups consisting of an alkyl group, a phenyl group, an amino group, and an azacycloalkyl group,
R² and R³ each independently indicates an alkyl group, and
R⁴ and R⁵ each independently indicates one of groups selected from groups consisting of an alkyl group, a phenyl group, and an alkyl carboxyl group.

8. The method as set forth in claim 3, wherein the molecules responsive to electric potential
